(19) United States
(12) Patent Application Publication
(10) Pub. No.: US 2007/0259393 A1
(43) Pub. Date: Nov. 8, 2007

(54) Plating media for the presumptive identification of Listeria sp, Listeria monocytogenes and Listeria ivanovii

(76) Inventor: Lawrence Restaino, Elburn, IL (US)

Correspondence Address: MARSHALL A. BURMEISTER, P. O. BOX 765, WILLIAMS BAY, WI, 53191, US

(21) Appl. No.: 11/415,510

(22) Filed: May 2, 2006

(51) Int. Cl. C12Q 1/04; C12N 1/20

(52) U.S. Cl. 435/34; 435/253.6; 435/252.1

(57) ABSTRACT

Isolation plating medium of a first color for the identification of Listeria sp. and Listeria monocytogenes and Listeria ivanovii containing a first chromogenic substrate that responds to phosphatidylinositol-specific phospholipase C enzymes to release precipitate of a second color into the medium and a second chromogenic substrate that responds to beta-glucosidase enzymes to release precipitate of a third color into the medium, and said first, second and third colors contrasting with each other.

The present invention relates to the presumptive identification of microorganisms, and in particular to the rapid, presumptive identification of Listeria sp., Listeria monocytogenes, and Listeria ivanovii.

### BACKGROUND OF THE INVENTION

Listeria monocytogenes is a human and animal pathogen that is widespread in nature. The organism is a transient constituent of intestinal flora excreted by 1-10% of healthy humans. It is an extremely hardy organism, and it can survive in the cold for many years in naturally infected sources. It has been isolated from a wide variety of foods, including dairy products, meats, and fish. All strains of Listeria monocytogenes are pathogenic. Listeria ivanovii, while not pathogenic for humans, is pathogenic for animals.

Food processors and public health officials have recognized Listeria sp. as an indicator of contamination of food, water and environmental conditions, and procedures have been established for monitoring foods, water and the environment for contamination requiring measurement of Listeria sp. Further, rapid detection and identification of the species Listeria monocytogenes and Listeria ivanovii are required for the diagnosis of patients by health care professionals. Accordingly, there is a need for a diagnostic tool that simultaneously detects and identifies Listeria sp. while separately detecting and identifying Listeria monocytogenes and Listeria ivanovii.

European Patent No. EP 0949266 entitled NOVEL POTENTIALLY FLUOROGENIC COMPOUNDS filed Mar. 23, 1998 naming Schabert and Restaino (the present inventor) as inventors, describes culture media that contain certain substrates that on contact with Listeria, and certain other bacteria, produce Phosphatidylinositol-Specific Phospholipase C enzymes, referred to herein as the PI-PLC enzyme, that react with the substrate to release a color matrix into the medium. Also, a Listeria monocytogenes detection system is described in a paper entitled ISOLATION AND DETECTION OF LISTERIA MONOCYTOGENES USING FLUOROGENIC AND CHROMOGENIC SUBSTRATES FOR PHOSPHATIDYLINOSITOL-SPECIFIC PHOSPHOLIPASE C by Restaino (the present inventor), Frampton, Irbe, Shabert and Spitz, Journal of Food Protection, Vol. 62, No. 3, 1999. This paper discloses a solid plating medium with a substrate that responds to contact with Listeria monocytogenes or Listeria ivanovii bacteria to produce the PI-PLC enzyme, and that enzyme reacts with the substrate to release a water insoluble precipitate of a distinctive color, thus causing colonies of Listeria monocytogenes and Listeria ivanovii to assume the color of the precipitate. Other strains of Listeria, namely, Listeria welshimeri, Listeria seeligeri, Listeria grayii and Listeria innocua, do not produce the PI-PLC enzyme on contact with this substrate, and accordingly, colonies of these bacteria are of the color of the plating medium. Since a count of Listeria sp. colonies requires summing the counts of Listeria monocytogenes colonies, Listeria ivanovii colonies and all other Listeria colonies, this medium requires the observance and counting of colonies that assume the color of the precipitate and colonies that are white. The colonies that are white are more difficult to determine as non-pathogenic Listeria sp. than the colonies that assume the color of the precipitate and are more likely to be erroneously counted. U.S. Pat. No. 6,228,606 entitled CULTURE MEDIUM FOR DETECTING PATHOGENIC BACTERIA OF THE GENUS LISTERIA AND METHOD FOR IDENTIFYING SAID BACTERIA of Jean-Pierre Facon et al. also describes a culture medium responsive to the PI-PLC enzyme produced by Listeria monocytogenes and Listeria ivanovii and in which colonies of other Listeria bacteria retain the color of the medium.

### SUMMARY OF THE INVENTION

It is an object of the present invention to provide a plating medium of a first color that reacts to Listeria monocytogenes and Listeria ivanovii to produce colonies of a second color, and also reacts to other strains of Listeria to produce colonies of a third color, so that the total number of colonies of the second and third colors is a measurement of Listeria sp. As stated above, a substrate is known that on contact with Listeria monocytogenes or Listeria ivanovii releases the enzyme PI-PLC which in turn cleaves the substrate to release a water insoluble precipitate of a second color, and the precipitate colors colonies of these bacteria, thus forming colonies of the second color. It is also known that on contact with certain other substrates, all strains of Listeria will produce beta-glucosidase enzymes, and the beta-glucosidase enzymes will cleave the substrate to release a water insoluble precipitate of a particular color. However, all substrates presently known that react to the strains of Listeria that do not produce PI-PLC also react to Listeria monocytogenes and Listeria ivanovii, and accordingly effect the color of Listeria monocytogenes and Listeria ivanovii colonies.

It is an object of the present invention to provide a solid plating medium of a first color that contains a first substrate that reacts to contact with Listeria monocytogenes and Listeria ivanovii to release PI-PLC enzymes which react with the first substrate to release precipitate of a second color, and a second substrate that on contact with any Listeria organism releases beta-glucosidase enzymes that react with the second substrate to release precipitate of a third color into the plating medium, the first, second and third colors contrasting with each other.

At the present time, the only substrate that is cleaved by the PI-PLC enzyme available commercially is 5-bromo-4-chloro-3-indoxyl-myo-inositol-1-phosphate. This substrate releases a dark blue precipitate responsive to a reaction with the PI-PLC enzyme, which is at the short wavelength region of the visible light range. While it is not intended to limit the invention to any particular colors, if this substrate is considered to be the first substrate, it becomes necessary to select a substrate that produces precipitate of a significantly longer wavelength than that of the first substrate (such as red) for the second beta-glucosidase reacting substrate in order to provide contrasting second and third colors. However, since Listeria monocytogenes and Listeria ivanovii produce PI-PLC enzymes on contact with the first substrate and beta-glucosidase enzymes on contact with the second substrate, and both substrates are present in the medium, precipitate of both substrates will be released into colonies of these bacteria, and the perceived color of the Listeria monocytogenes and Listeria ivanovii colonies will be a blend of the second and third colors. As a result, the contrast between the Listeria monocytogenes/Listeria ivanovii colonies and the other Listeria colonies tends to be materially reduced and the medium tends to be more difficult to read than it would be if the Listeria monocytogenes/Listeria ivanovii colonies did not blend with the color of the other Listeria colonies.

The inventor has found that the contrast between the Listeria monocytogenes/Listeria ivanovii colonies and the colonies of other Listeria bacteria can be preserved by reducing the concentration of the second substrate in the medium to a value that does not materially effect the color of the Listeria monocytogenes/Listeria ivanovii colonies, however, the intensity of the color of the colonies of the other Listeria species is strong enough to visually read against the color of the medium. In order to increase the intensity of the color of colonies of Listeria other than Listeria monocytogenes/Listeria ivanovii to an acceptable level without materially affecting the color of Listeria monocytogenes and Listeria ivanovii colonies, the inventor has added a third substrate to the medium that, like the second substrate, is responsive to Listeria sp. to produce beta-glucosidase enzymes which react with the third substrate to produce a water insoluble precipitate of a fourth color that is in the long wave-length portion of the visible light range but different than the third color. The inventor has found that the addition of a concentration of the third substrate approximately equal to the concentration of the second substrate results in a blending of the third and fourth colors in each colony producing the beta-glucosidase enzyme, thus producing a composite color with a wavelength between the wavelengths of the third and fourth colors. In colonies of Listeria monocytogenes or Listeria ivanovii, the second, third and fourth colors blend, but the intensity of the second color is so much greater than the intensity of the third and fourth colors that it dominates the blend and the color of these colonies remains essentially the second color.

Hence, it is an object of the present invention to provide a solid culture plating medium of a first color capable of growing colonies of Listeria sp. that contains a first substrate that on contact reacts to Listeria monocytogenes and Listeria ivanovii bacteria to release PI-PLC enzymes that react with the first substrate to release precipitate of a second color into colonies of Listeria monocytogenes and Listeria ivanovii in the plating medium, and a second substrate that on contact with any Listeria organism releases into the medium beta-glucosidase enzymes that react with the second substrate to release precipitate of a third color into Listeria sp. colonies on the plating medium, the quantity of the second substrate being insufficient for the precipitate of the second substrate to materially effect the color of Listeria monocytogenes and Listeria ivanovii colonies, and a third substrate that on contact with any Listeria organism releases into the medium beta-glucosidase enzymes that react with the third substrate to release precipitate of a fourth color into Listeria sp. colonies on the plating medium, the quantity of the third substrate being insufficient for the precipitate of the third substrate to materially effect the color of the Listeria monocytogenes and Listeria ivanovii colonies, the third and fourth colors blending to a fifth color that contrasts with the first and second colors.

### DETAILED DESCRIPTION OF THE INVENTION

It is necessary that Listeria sp. bacteria consume nutrients and grow in order for the bacteria to secrete enzymes. Hence the plating medium must have a rich nutrient base. In order to promote the growth of the various strains of Listeria sp. bacteria, the plating media of the present invention includes one or more of the ingredients proteose peptone, Lab Lemco powder, yeast extract, tryptone, casamino acids and glucose. In the preferred medium described in this specification, all of these ingredients are included in the plating medium and form the nutrient base.

In any plating medium, the growth of cells of bacteria other than the bacteria of interest complicates or completely frustrates reading of the plate, and hence it is desirable to include inhibitors in the medium. The media of the present invention suppress unwanted microorganisms that react with a substrate of the media releasing either beta glucosidase or PI-PLC enzymes. Accordingly, all strains of Bacillus are inhibited, since all strains of Bacillus are beta-glucosidase positive and Bacillus cereus and Bacillus thuringiensis are PI-PLC positive. For this purpose, the media of the present invention contain one or more of the ingredients naladixic acid, sodium salt, lithium chloride, ceftazidime, and the third and fourth generation of cephalosporins. The preferred plating medium contains nalidixic acid, sodium salt.

Some species of yeasts and molds are PI-PLC positive, and thus the medium contains an ingredient to inhibit the growth of yeast and molds. The preferred plating medium contains cycloheximide for this purpose.

Enterococcus sp. also reacts to the substrates used in the present plating media to release beta-glucosidase enzymes. Lithium chloride inhibits growth of Enterococcus sp., and also inhibits growth of gram negative bacteria. Accordingly, the preferred medium of the present invention includes lithium chloride.

Staphylococcus sp. also reacts to the substrates used in the present plating media to release beta-glucosidase enzymes. Accordingly, the preferred medium of the present invention includes ceftazidime pentahydrate to inhibit growth of Staphylococcus sp.

The chromogenic substrate that releases color into a medium responsive to the presence of phosphatidylinositol-specific phospholipase C in the preferred medium is 5-bromo-4-chloro-3-indoxyl-myo-inositol-1-phosphate. At the present time, this is the only chromogenic substrate commercially available that reacts to the PI-PLC enzyme. It reacts with the PI-PLC enzyme to release a dark blue water insoluble precipitate.

Ingredients that permit the activation of the enzyme phosphatidylinositol-specific phospholipase C in plating media are bovine serum and powdered silicates. In the preferred embodiment, this ingredient is bovine serum.

The plating media also contains at least one chromogenic substrate that releases color into the medium responsive to the presence of beta-glucosidase enzymes, the released color contrasting with the color released by the reaction of PI-PLC enzymes with the substrate referred to above. In the preferred medium, two substrates responsive to the enzyme beta-glucosidase are included in the medium, namely, 5-bromo-6-chloro-3indoxyl-beta-D-glucopyranoside (referred to herein as magenta-β-D-glucoside) and 6-chloro-3-indoxyl-beta-D-glucopyranoside (referred to herein as salmon-β-D-glucoside).

The plating media also contains at least one ingredient to maintain the pH of the medium in a suitable range, namely, potassium phosphate (monobasic) and/or sodium phosphate (dibasic). In the preferred embodiment, potassium phosphate is used in the medium.

An ingredient must be added to solidify the mixture. In the preferred mixture, this ingredient is agar.

### EXAMPLE 1

The preferred embodiment of a plating medium according to the present invention consists of the ingredients set forth in Table 1.

TABLE 1

| CHEMICAL | SUPPLIER | GRAMS/LITER |
|---|---|---|
| Proteose peptone | Difco | 3.00 |
| Tryptone | Difco | 9.60 |
| Casamino acids | Difco | 6.0 |
| Lab Lemco powder | Oxoid | 5.00 |
| Glucose | Anyplace | 2.5 |
| Yeast extract | Difco | 7.00 |
| Potassium phosphate | Anyplace | 4.5 |
| (Dibasic} | | |
| Lithium chloride | Anyplace | 9.0 |
| Cycloheximide | Anyplace | 0.20 |
| Nalidixic acid, | Anyplace | 0.01 |
| sodium salt | | |
| Salmon-β-D- | Biosynth | 0.030 |
| glucoside | | |
| Magenta-β-D- | Biosynth | 0.033 |
| glucoside | | |
| Agar | Difco | 15.00 |
| Titanium oxide (IV) | Sigma | 3.0 |
| Bovine albumin | Serologicals | 3.0 |
| Ceftazidime | Glaxo Wellcome | 0.035 |
| pentahydrate | | |
| 5-bromo-4-chloro-3- | Biosynth | 0.29 |
| indoxyl-myo-inositol- | | |
| 1-phosphate. | | |

Prior to the preparation of the plating medium, the ingredients are mixed into four portions. The first portion contains proteose peptone, tryptone, potassium phosphate (dibasic), and nalidixic acid. The second portion contains casamino acids, yeast extract, Lab Lemco Powder, salmon-β-D glucoside and magenta-β-D glucoside. The third portion contains glucose, lithium chloride, cycloheximide and agar. The fourth portion contains the remaining ingredients, namely, titanium oxide (IV), bovine albumin, ceftazidime pentahydrate and 5-bromo-4-chloro-3-indoxyl-myo-inositol-1-phosphate, each of which is maintained separately under its prescribed stage conditions until the plating medium is to be produced.

The composition is prepared by first mixing the first three portions set forth above plus titanium oxide (IV) under sterile conditions. Thereafter the remaining three components of the fourth portion, bovine albumin, ceftazidime pentahydrate and 5-bromo-4-chloro-3-indoxyl-myo-inositol-1-phosphate, are added to the mixture. The mixture is then placed in petri dishes and stored under proper conditions overnight. It has a white color.

The bacterial strains indicated in Table 2 were applied to the Petri dishes referred to above and incubated at 35 degrees Celsius for a period of 48 hours. Thereafter, the white surfaces of the plating media in the petri dishes were observed under white light, and produced the results set forth in Table 2 below.

TABLE 2

| | Number | |
|---|---|---|
| | of | |
| Bacteria | Strains | Colonial Morphology |
| Listeria monocytogenes | 39 | Convex, 1-2 mm., blue-green |
| | | to blue-violet, ±precipitate |
| Listeria ivanovii | 4 | Convex, 1-1.5 mm, dark |
| | | blue-green, large precipitate |
| Listeria innocua | 6 | Convex, 1-2 mm., pink, no |
| | | precipitate |
| Listeria welshimerti | 2 | Convex, 1-2 mm., pink, no |
| | | precipitate |
| Listeria seeligeri | 1 | Convex, 1-2 mm., pink, no |
| | | precipitate |
| Listeria grayii | 1 | Convex, 1-2 mm., pink, no |
| | | precipitate |
| Bacillus | 3 | No growth |
| cereus/thuringiensis | | |
| Enterococcus spp.* | 3 | No growth |
| Gram positive spp.** | — | No growth |
| Gram negative spp.*** | — | No growth |
| Yeasts.**** | 3 | No growth |

*Enterococcus faecalis, Enterococcus faecium, and Enterococcus avium.
**Includes, Bacillus circulans, and Bacillus subtilis, Staphylococcus aureus, Staphylococcus epidermidis, Staphylococcus saprolyticus, Lactobacillus acidophilus, Lactobacillus plantarum; and Pediococcus cerevisiae.
***Includes Escherichia coli (2 strains), Escherichia coli 0157:H7 (1 strain), Enterobacter aerogenes, Citrobacter freundii, Shigella sonnei, Morganella morganii, Providensia alcalifaciens, Pantoea aggomerans, Enterobacter sakazakii, Klebsiella pneumoniae, and Klebsiella ozanae.
****Zygosaccharomyces bailii, and Zygosaccharomyces rouxii; Candida albicans.

As indicated in Table 2, all of the strains of Listeria produced colonies in the plating media of significant size to facilitate detection and counting. Further, colonies of Listeria monocytogenes and Listeria ivanovii produced colonies of blue-green color and the other strains of Listeria produced colonies of pink color. Since the color of the medium is white, all Listeria colonies are readily distinguished from the medium by the naked eye, and colonies of Listeria monocytogenes and Listeria ivanovii are readily distinguished from other strains of Listeria.

Differentiation of the Listeria monocytogenes and Listeria ivanovii cells has been achieved by selecting the color of the precipitate released by the three substrates in response to contact with an appropriate enzyme, and controlling the relative intensity of the Listeria monocytogenes/Listeria ivanovii cells with respect to the cells of other Listeria strains. In the preferred plating medium, the inventor has provided a sufficient concentration of the first substrate (5-bromo-4-chloro-3-indoxyl-myo-inositol-1-phosphate) that responds to the PI-PLC enzyme to color colonies of Listeria monocytogenes and Listeria ivanovii to an intensity that is easily observed and a color that is located near the upper frequency end of the visible range of the electromagnetic spectrum. A second substrate (magenta-β-D-glucoside) is also incorporated in the medium to react to an enzyme produced by all strains of Listeria (beta-glucosidase), but this substrate releases precipitate of a color at the lower end of the visible electromagnetic spectrum (magenta). Both PI-PLC enzymes and the beta-glucosidase enzymes are present in Listeria monocytogenes/Listeria ivanovii colonies, and hence precipitate of blue-green and precipitate of magenta are present in these colonies and light waves reflected therefrom. By restricting the intensity of the magenta color in the Listeria monocytogenes/Listeria ivanovii colonies, the observer perceives these colonies to be the color of the first substrate.

However, under these conditions, the color of the colonies that contain only precipitate of the magenta color (the colonies of the strains of Listeria other than Listeria monocytogenes and Listeria ivanovii) have insufficient precipitate to permit the observer to readily distinguish these colonies from the color of the medium. The addition of a low concentration of a third substrate to the medium that responds to the enzyme beta-glucosidase to release precipitate of a fourth color of a wavelength relatively near to that of the third color results in colonies of strains of Listeria other than Listeria monocytogenes and Listeria ivanovii appearing brightly colored to the eye of the observer without significantly changing the color of the colonies of Listeria monocytogenes and Listeria ivanovii. In the preferred plating media describe above, the third substrate is Salmon-β-D-glucoside which produces salmon colored precipitate, and the concentration of the third substrate in the medium is similar to the concentration of the second substrate.

While not desiring to be bound by any particular theory, it is believed that the eye of the observer receives two electromagnetic waves of slightly different wavelength from the colonies of strains of Listeria other than Listeria monocytogenes and Listeria ivanovii, and the observer adds these electromagnetic waves algebraically to produce a composite or blended electromagnetic wave of significantly greater amplitude than either of the received electromagnetic waves. Hence, the observer perceives the color of the colonies of these bacteria to be significantly brighter than the color the colonies would be if either the second or third substrate were omitted from the medium.

However, the color of the colonies of Listeria monocytogenes and Listeria ivanovii are not significantly changed by the presence of the third substrate in the medium. These colonies contain precipitate from all three substrates in the same proportions as the concentration of the substrates in the medium, and hence each of these colonies reflects three electromagnetic wave beams to the eye of the observer. The observer makes an algebraic summation of the three electromagnetic waves, and because of the fact that the magenta and salmon waves are of different wavelengths and significantly smaller amplitudes than the blue-green electromagnetic waves of the first substrate, the observer does not perceive a significant distortion in the color of the Listeria monocytogenes and Listeria ivanovii colonies by the addition of the second and third substrates to the medium.

### EXAMPLE 2

In Example 2, two changes are made in the ingredients listed in Table 1, namely, the third substrate, salmon beta-D-glucoside, is omitted from the mixture, and the concentration of the second substrate, magenta beta-D-glucoside, is doubled to 0.066 grams/liter. This change in formulation produces brightly colored colonies of strains of Listeria other than Listeria monocytogenes and Listeria ivanovii of the magenta color.

However, this plating medium also produces bright colonies of Listeria monocytogenes and Listeria ivanovii, but not of a blue green color as in Example 1, but rather of a green color. Colonies of Listeria monocytogenes and Listeria ivanovii contain precipitate of both dark blue from the first substrate and magenta from the second substrate, and these colonies reflect beams of both of these colors to the eye of the observer to produce a composite or blended image of an intermediate wavelength, namely green. The green colored Listeria monocytogenes and Listeria ivanovii colonies may be detected and counted by observing the surface of the plating medium, and the magenta colored colonies of the other strains of Listeria may also be observed and counted to determine a count for Listeria sp. Results similar to the results of Table 2 may be obtained using the medium of Example 2.

Variations in the plating medium set forth above will become apparent to those skilled in the art. It is therefore intended that this invention be not limited to the foregoing specification, but rather only to the appended claims

The invention claimed is:

1. Isolation plating medium of a first color for the identification of Listeria sp. and Listeria monocytogenes and Listeria ivanovii consisting essentially of a mixture of (1) nutrients that promote growth of cells of Listeria sp. under suitable environmental conditions for growth, (2) at least one ingredient that inhibits the growth of microorganisms other than Listeria sp. under suitable environmental conditions for growth, (3) a first chromogenic substrate that responds to contact with phosphatidylinositol-specific phospholipase C enzymes to release a water insoluble precipitate of a second color into the medium at the contact location (4) a second chromogenic substrate that responds to contact with beta-glucosidase enzymes to release a water insoluable precipitate of a third color at the contact location, Listeria monocytogenes and Listeria ivanovii bacteria producing phosphatidylinositol-specific phospholipase C enzymes and beta-glucosidase enzymes on contact with a substrate and Listeria sp. bacteria producing beta-glucosidase enzymes on contact with a substrate, and said first, second and third colors contrasting with each other, and (5) an ingredient for thickening the mixture in sufficient quantity_to solidify the mixture, whereby Listeria sp. bacteria will produce colonies in the medium of a color different than the color of the medium, and Listeria monocytogens and Listeria ivanovii bacteria contacting the medium will produce colonies in the medium of a different color than the medium and other strains of Listeria.

2. The medium of claim 1 wherein the nutrients that promote growth of cells of Listeria sp. consists of one or more members of the class proteose peptone, tryptone, casimino acids, meat extract powder, glucose, and yeast extract.

3. The medium of claim 1 wherein the one or more ingredients that inhibit growth of microorganisms other than Listeria sp. consists of one or more members of the group lithium chloride, nalidixic acid, sodium salt, cycloheximide, sodium salt, and ceftazidime pentahydrate.

4. The medium of claim 1 wherein the first chromogenic substrate that releases precipitate of a first color responsive to the contact of phosphatidylinositol-specific phospholipase C enzymes is 5-bromo-4-chloro-3-indoxyl-myo-inositol-1-phosphate.

5. The medium of claim 1 wherein the second chromogenic substrate that releases precipitate of a second color responsive to the contact of the beta-glucosidase enzyme consists of one or more members of the group salmon-beta-D-glucoside and magenta-beta-D-glucoside.

6. The medium of claim 1 wherein the ingredient for thickening the mixture to solidify the mixture is agar.

7. The medium of claim 1 including a third chromogenic substrate that responds to contact with the beta-glucosidase enzyme to release a water insoluable precipitate of a fourth color at the contact location, said second color contrasting with the third and fourth colors and with the first color, the wavelengths of the third and fourth colors being closer to each other than to the wavelength of the second color, the third and fourth colors blending to produce a fifth color which contrasts with the first and second colors, and the concentration of the third and fourth substrates in the medium being insufficient to materially effect the second color.

8. The medium of claim 7 wherein the first chromogenic substrate is 5-bromo-4-chloro-3-indoxyl-myo-inositol-1-phosphate.

9. The medium of claim 8 wherein the second chromogenic substrate is magenta-beta-D-glucoside and the third chromogenic substrate is salmon-beta-D-glucoside.

10. The medium of claim 9 wherein the medium includes a sufficient quantity of titanium oxide (IV) to be perceived as substantially white.

11. The medium of claim 1 wherein the concentrations of the first and second chromogenic substrates are sufficient to produce colonies with colors of similar intensity, whereby the color of the colonies of Listeria monocytogenes and Listeria ivanovii assume a color which is a blend of the colors of the precipitation from the first and second substrates.

12. The medium of claim 11 wherein the first chromogenic substrate releases precipitate with a color at the shorter wavelength end of the visible electromagnetic spectrum, and the second chromogenic substrate releases precipitate with a color at the longer wavelength end of the electromagnetic spectrum, whereby the color of the colonies of Listeria monocytogenes and Listeria ivanovii assume a color which is a blend of the colors of the precipitation from the first and second substrates, and the colonies of the other Listeria strains assume the color of the precipitate from the second substrate.

13. The medium of claim 11 wherein the first chromogenic substrate is 5-bromo-4-chloro-3-indoxyl-myo-inositol-l-phosphate, and the second chromogenic substrate is magenta-beta-D-glucoside, whereby the color of the colonies of Listeria monocytogenes and Listeria ivanovii assume a greenish color which is a blend of the colors of the precipitation from the first and second substrates, and the colonies of the other Listeria strains assume a magenta color.
